(19) United States
(12) Patent Application Publication
(10) Pub. No.: US 2024/0368165 A1
(43) Pub. Date: Nov. 7, 2024

(54) SOLID STATE FORMS OF HYDROCHLORIDE SALT OF ((1S,2S,4R)-4-{4-[(1S)-2,3-DIHYDRO-1H-INDEN-1-YLAMINO]-7H-PYRROLO[2,3-D]PYRIMIDIN-7-YL}-2-HYDROXYCYCLOPENTYL)METHYL SULFAMATE

(71) Applicant: Takeda Pharmaceutical Company Limited, Osaka (JP)

(72) Inventors: Koya KIMOTO, Osaka (JP); Marianne LANGSTON, Cambridge, MA (US)

(21) Appl. No.: 18/254,550

(22) PCT Filed: Nov. 23, 2021
(86) PCT No.: PCT/IB2021/060892

§ 371 Date: May 25, 2023

Related U.S. Application Data
(60) Provisional application No. 63/118,449, filed on Nov. 25, 2020.

(51) Int. Cl. C07D 487/04; A61K 31/519

(52) U.S. Cl. CPC C07D 487/04; A61K 31/519

(57) ABSTRACT

Disclosed herein are solid state forms of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride, compositions thereof, methods for their preparation, and methods for their use.

### FIELD

The present invention discloses certain new solid state forms of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate, processes for preparing such forms, pharmaceutical compositions comprising them, and the use of such forms in therapy.

### BACKGROUND

The post-translational modification of proteins by ubiquitin-like molecules (ubls) is an important regulatory process within cells, playing key roles in controlling many biological processes including cell division, cell signaling and the immune response. Ubls are small proteins that are covalently attached to a lysine on a target protein via an isopeptide linkage with a C-terminal glycine of the ubl. The ubiquitin-like molecule alters the molecular surface of the target protein and can affect such properties as protein-protein interactions, enzymatic activity, stability and cellular localization of the target.

Ubiquitin and other ubls are activated by a specific E1 enzyme which catalyzes the formation of an acyl-adenylate intermediate with the C-terminal glycine of the ubl. The activated ubl molecule is then transferred to the catalytic cysteine residue within the E1 enzyme through formation of a thioester bond intermediate. The E1-ubl intermediate and an E2 associate, resulting in a thioester exchange wherein the ubl is transferred to the active site cysteine of the E2. The ubl is then conjugated to the target protein, either directly or in conjunction with an E3 ligase, through isopeptide bond formation with the amino group of a lysine side chain in the target protein.

Targeting E1 activating enzymes provides a unique opportunity to interfere with a variety of biochemical pathways important for maintaining the integrity of cell division and cell signaling. E1 activating enzymes function at the first step of ubl conjugation pathways; thus, inhibition of an E1 activating enzyme will specifically modulate the downstream biological consequences of the ubl modification. As such, inhibition of these activating enzymes, and the resultant inhibition of downstream effects of ubl-conjugation, represents a method of interfering with the integrity of cell division, cell signaling, and several aspects of cellular physiology which are important for disease mechanisms. Thus, E1 enzymes such as UAE, NAE, and SAE, as regulators of diverse cellular functions, are potentially important therapeutic targets for the identification of novel approaches to treatment of diseases and disorders.

Langston S. et al., Intl. App. Pub. No. WO 07/092213 and Langston S. et al, U.S. App. Pub. No. 2007/0191293, which are hereby incorporated by reference in their entirety, disclose compounds which are effective inhibitors of E1 activating enzymes, particularly NAE. The compounds are useful for inhibiting E1 activity in vitro and in vivo and are useful for the treatment of disorders of cell proliferation, particularly cancer, and other disorders associated with E1 activity. One class of compounds described in Langston et al. are 4-substituted ((1S, 2S, 4R)-2-hydroxy-4-{7H-pyrrolo[2,3-d]pyrimidin-7-yl}cyclopentyl)methyl sulfamates. Armitage I. et al., U.S. App. Pub. No. 2009/0036678, which is hereby incorporated by reference in its entirety, discloses methods for the preparation of ((1S, 2S, 4R)-2-hydroxy-4-{7H-pyrrolo[2,3-d]pyrimidin-7-yl}cyclopentyl)methyl sulfamates, including ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate. This compound has been reported to be a selective NAE inhibitor. See, e.g., Soucy, T. A., et al., Nature, 2009, 458, 732-737 (which refers to the compound as MLN4924, also known as TAK-924, or pevonedistat).

((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (I) is described in Intl. App. Pub. No. WO 07/092213, U.S. App. Pub. No. 2007/0191293, and U.S. App. Pub. No. 2009/0036678.

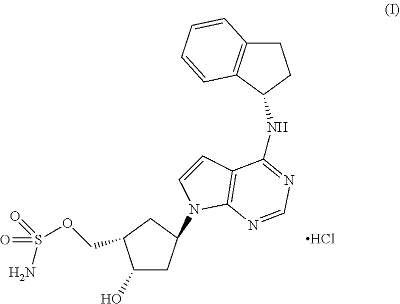 [SMILES: NS(=O)(=O)OC[C@@H]1C[C@@H](n2ccc3c(N[C@H]4CCc5ccccc54)ncnc32)C[C@@H]1O ; Cl]

U.S. Appl. Pub. No. 2011/0021544 which is incorporated by reference in its entirety discloses the hydrochloride salt of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (I), crystalline forms thereof, solvates thereof, and methods of making them. U.S. Appl. Pub. No. 2011/0021544 further discloses crystalline forms Form 1, Form 2, Form 3A, Form 3B, Form 3C, Form 5, and Form 7 of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (I), methods of making these forms, pharmaceutical compositions and method of treatment comprising administering these forms.

Intl. App. Pub. No. PCT/US18/51940 which is incorporated by reference in its entirety, discloses co-crystal forms of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (I), methods of making, pharmaceutical compositions and method of treatment comprising administering these cocrystal forms. These applications additionally disclose pharmaceutical compositions containing these compounds, and methods for the treatment or therapy of diseases, disorders, or conditions associated with E1 activating enzymes, particularly NAE, including proliferative diseases such as cancer.

The large-scale manufacturing of a pharmaceutical composition poses many challenges to the chemist and chemical engineer. While many of these challenges relate to the handling of large quantities of reagents and control of large-scale reactions, the handling of the final product poses special challenges linked to the nature of the final active product itself. Not only must the product be prepared in high yield, be stable, and capable of ready isolation, the product must possess properties that are suitable for the types of pharmaceutical preparations in which they are likely to be ultimately used. The stability of the active ingredient of the pharmaceutical preparation must be considered during each step of the manufacturing process, including the synthesis, isolation, bulk storage, pharmaceutical formulation and long-term formulation. Each of these steps may be impacted by various environmental conditions of temperature and humidity.

The pharmaceutically active substance used to prepare the pharmaceutical compositions should be as pure as possible and its stability on long-term storage must be guaranteed under various environmental conditions. These properties are absolutely essential to prevent the appearance of unintended degradation products in pharmaceutical compositions, which degradation products may be potentially toxic or result simply in reducing the potency of the composition.

A primary concern for the manufacture of large-scale pharmaceutical compounds is that the active substance should have a stable crystalline morphology to ensure consistent processing parameters and pharmaceutical quality. If an unstable crystalline form is used, crystal morphology may change during manufacture and/or storage resulting in quality control problems, and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” While polymorphs of a given substance have the same chemical composition, they may differ from each other with respect to one or more physical properties, such as solubility and dissociation, true density, melting point, crystal shape, compaction behavior, flow properties, and/or solid state stability.

As described generally above, the polymorphic behavior of drugs can be of great importance in pharmacy and pharmacology. The differences in physical properties exhibited by polymorphs affect practical parameters such as storage stability, compressibility and density (important in formulation and product manufacturing), and dissolution rates (an important factor in determining bio-availability). Differences in stability can result from changes in chemical reactivity (e.g., differential oxidation, such that a dosage form discolors more rapidly when it is one polymorph than when it is another polymorph) or mechanical changes (e.g., tablets crumble on storage as a kinetically favored polymorph converts to thermodynamically more stable polymorph) or both (e.g., tablets of one polymorph are more susceptible to breakdown at high humidity). In addition, the physical properties of the crystal may be important in processing: for example, one polymorph might be more likely to form solvates that cause the solid form to aggregate and increase the difficulty of solid handling, or might be difficult to filter and wash free of impurities (i.e., particle shape and size distribution might be different between one polymorph relative to other).

While drug formulations having improved chemical and physical properties are desired, there is no predictable means for preparing new drug forms (e.g., polymorphs) of existing molecules for such formulations. These new forms would provide consistency in physical properties over a range of environments common to manufacturing and composition usage. Thus, there is a need for new drug forms that are useful for inhibiting E1 activity in vitro and in vivo, and are useful for the treatment of disorders of cell proliferation, particularly cancer, and other disorders associated with E1 activity, as well as having properties suitable for large-scale manufacturing and formulation.

### SUMMARY

The present disclosure is directed to novel solid state forms of the hydrochloride salt of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (I), referred to as Compound 1 in this application.

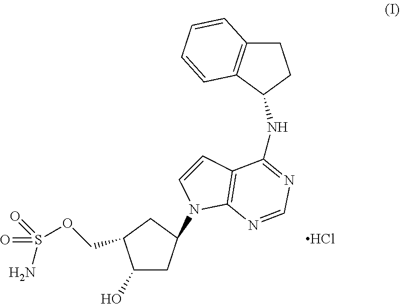 [SMILES: NS(=O)(=O)OC[C@@H]1C[C@@H](n2ccc3c(N[C@H]4CCc5ccccc54)ncnc32)C[C@@H]1O ; Cl]

In some embodiments, the present disclosure provides a solid form of Compound 1, referred to as Form 9.

In some other embodiments, present disclosure provides a solid form of Compound 1, referred to as Form 12.

The disclosure also discloses methods of making these solid-state forms. The present disclosure further discloses pharmaceutical compositions comprising these solid-state forms and methods of uses of these forms for the treatment of a variety of diseases, disorders or conditions as described herein.

The present invention shall be more fully discussed with the aid of the following figures and detailed description below.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the present invention.

### BRIEF DESCRIPTION OF THE DRAWINGS

FIG. 1A is a powder X-ray diffractogram (XRPD) of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9.

FIG. 1B is a differential scanning calorimetry (DSC) profile for ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9.

FIG. 1C is a thermal gravimetric analysis (TGA) profile for ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9.

FIG. 2A is a powder X-ray diffractogram (XRPD) of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12.

FIG. 2B is a differential scanning calorimetry (DSC) profile for ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12.

FIG. 2C is a thermal gravimetric analysis (TGA) profile for ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12.

FIG. 3A is an XRPD overlay of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9 after accelerated storage.

FIG. 3B is an XRPD overlay of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12 after accelerated storage.

### DETAILED DESCRIPTION

As used herein, the term “about” when used in reference to a degree 2θ value refers to the stated value±0.2 degree 2θ. One of skill in the art will appreciate that changes in the particular XRPD acquisition parameters will affect the XRPD pattern and specific values of degrees 2θ obtained.

The term “substantially as shown in” when referring to an X-ray powder diffraction pattern, differential scanning calorimetry pattern or thermal gravimetric analysis means that a pattern that is not necessarily identical to those depicted herein, but that falls within the limits of experimental error or deviations, when considered by one of ordinary skill in the art.

As used herein, the term “polymorph” refers to any of the different crystal structures in which a compound can crystallize.

As used herein, the term “solvate” refers to a crystal form with either a stoichiometric or non-stoichiometric amount of solvent incorporated into the crystal structure. Similarly, the term “hydrate” refers specifically to a crystal form with either a stoichiometric or non-stoichiometric amount of water incorporated into the crystal structure.

It has been found that ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride can exist in a variety of solid forms.

U.S. Pat. No. 9,187,482 B2, which is incorporated by reference in its entirety, discloses Form 1, Form 2, Form 3A, Form 3B, Form 3C, Form 5, and Form 7 of ((1S,2S,4R)-4-{4-[(1S)-2,3-dihydro-1H-inden-1-ylamino]-7H-pyrrolo[2,3-d]pyrimidin-7-yl}-2 hydroxycyclopentyl)methyl sulfamate hydrochloride (I), methods of making these forms, pharmaceutical compositions and method of treatment comprising administering these forms.

In an embodiment, the present disclosure is directed to novel solid state forms of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride. These forms have properties that are useful for large-scale manufacturing, pharmaceutical formulation, and storage. Two of these new solid forms are referred to hereafter as “Form 9” and “Form 12.”

In some embodiments, the present disclosure provides a solid form of Compound 1, referred to as Form 9. In some embodiments, present disclosure provides a solid form of Compound 1, referred to as Form 12.

### Form 9

Form 9 is a solid state form of Compound 1 that can be identified by X-ray powder diffraction (XRPD).

In an embodiment, polymorphic Form 9 has an X-ray powder diffraction pattern comprising characteristic peaks plus or minus 0.2 degrees at 2θ angles of 15.60°, 17.16° and 18.30°.

In another embodiment, polymorphic Form 9 has an X-ray powder diffraction pattern comprising characteristic peaks plus or minus 0.2 degrees at 2θ angles of 15.60°, 17.16°, 18.30° and 24.10°.

In an embodiment, polymorphic Form 9 has an X-ray powder diffraction pattern further comprising at least one of characteristic peaks plus or minus 0.2 degrees at 2θ angles of 12.55°, 20.39°, 22.45°, or 27.63°.

It should be understood that relative intensities can vary depending on a number of factors, including sample preparation, mounting, and the instrument and analytical procedure and settings used to obtain the spectrum. As such, the peak assignments listed herein are intended to encompass variations of plus or minus 0.2 degrees 2θ.

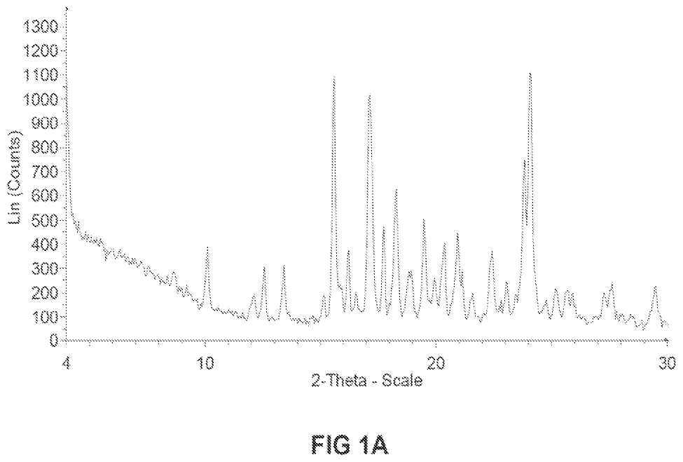

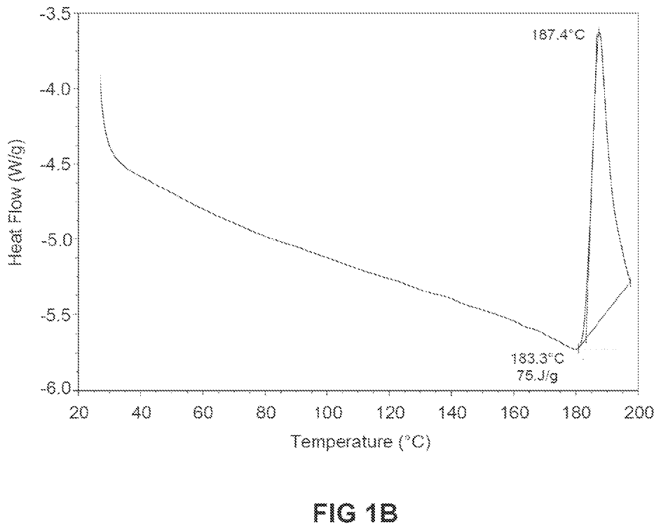

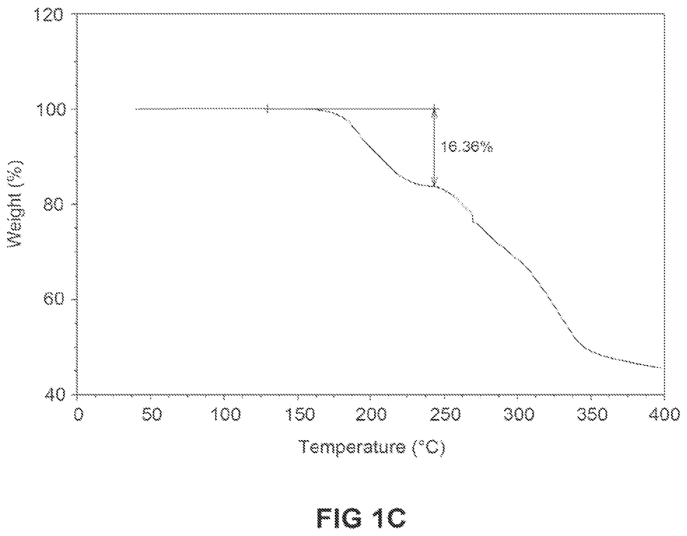

In another embodiment of the invention, Form 9 can be characterized by at least one of the following features (i)-(ii):
i. at least one, at least two, at least three, at least four, at least five, at least six, at least seven, at least eight, at least nine, at least ten, at least eleven or all of the X-ray powder diffraction peaks shown in Table 1 using CuKα radiation;
ii. an X-ray powder diffraction pattern substantially similar to FIG. 1A obtained using CuKα radiation.

TABLE 1

| Diffraction Angle | d value |
|---|---|
| ° (2θ) ± 0.2 | Angstrom |
| 8.63 | 10.244 ± 0.237 |
| 10.10 | 8.754 ± 0.173 |
| 12.09 | 7.316 ± 0.121 |
| 12.55 | 7.049 ± 0.112 |
| 13.40 | 6.604 ± 0.098 |
| 15.14 | 5.847 ± 0.077 |
| 15.60 | 5.677 ± 0.072 |
| 16.23 | 5.458 ± 0.067 |
| 16.55 | 5.353 ± 0.064 |
| 17.16 | 5.163 ± 0.060 |
| 17.74 | 4.995 ± 0.056 |
| 18.30 | 4.844 ± 0.052 |
| 18.89 | 4.693 ± 0.049 |
| 19.50 | 4.549 ± 0.046 |
| 19.95 | 4.447 ± 0.044 |
| 20.39 | 4.352 ± 0.042 |
| 20.95 | 4.238 ± 0.040 |
| 21.59 | 4.113 ± 0.038 |
| 22.45 | 3.957 ± 0.035 |
| 23.06 | 3.853 ± 0.033 |
| 23.84 | 3.729 ± 0.031 |
| 24.10 | 3.689 ± 0.030 |
| 24.79 | 3.589 ± 0.029 |
| 25.67 | 3.468 ± 0.027 |
| 25.93 | 3.434 ± 0.026 |
| 27.29 | 3.265 ± 0.023 |
| 27.63 | 3.226 ± 0.023 |
| 28.09 | 3.174 ± 0.022 |
| 28.51 | 3.128 ± 0.021 |
| 29.51 | 3.025 ± 0.020 |

In an embodiment, Form 9 can be characterized by the differential scanning calorimetry profile (DSC) shown in FIG. 1B. The DSC graph plots the heat flow as a function of temperature from a sample, characterized by an exothermic peak at about 187.4° C. with onset at 183.3° C.

In an embodiment, Form 9 can be characterized by TGA profile, as shown in FIG. 1C. TGA analysis shows a 16.4% w/w mass loss between 150-253° C.

Form 9 described herein has a solubility of about 7.8 mg/ml at a pH of 2.33.

One embodiment is directed to crystalline Form 9, wherein the crystalline form is characterized by at least two of the following features (i)-(iv):
(i) at least three of the X-ray powder diffraction peaks shown in Table 1;
(ii) an X-ray powder diffraction pattern substantially similar to FIG. 1A;
(iii) a differential scanning calorimetry (DSC) profile having an exotherm range of about 182° C. to about 198° C., with an onset of about 183.3° C.; or
(iv) a thermogravimetric analysis (TGA) profile that shows a 16.4% w/w mass loss between 150-253° C.

### Form 12

Form 12 is a solid state form of Compound 1 that can be identified X-ray powder diffraction (XRPD).

In an embodiment, polymorphic Form 12 has an X-ray powder diffraction pattern comprising characteristic peaks plus or minus 0.2 degrees at 2θ angles of 8.69°, 16.55°, 16.75 and 18.64°.

In another embodiment, polymorphic Form 12 has an X-ray powder diffraction pattern comprising characteristic peaks plus or minus 0.2 degrees at 2θ angles of 8.69°, 16.55°, 16.75, 17.51°, 18.64, 22.11° and 23.65°.

In an embodiment, polymorphic Form 12 has an X-ray powder diffraction pattern further comprising at least one of characteristic peaks plus or minus 0.2 degrees at 2θ angles of 19.02°, 21.17°, 25.90, 26.48° and 28.97.

It should be understood that relative intensities can vary depending on a number of factors, including sample preparation, mounting, and the instrument and analytical procedure and settings used to obtain the spectrum. As such, the peak assignments listed herein are intended to encompass variations of plus or minus 0.2 degrees 2θ.

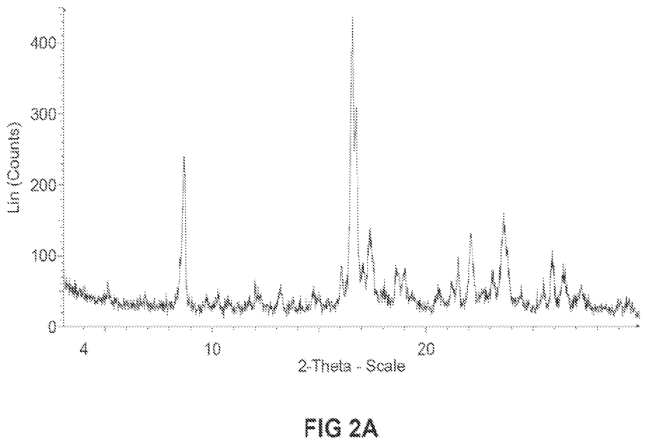

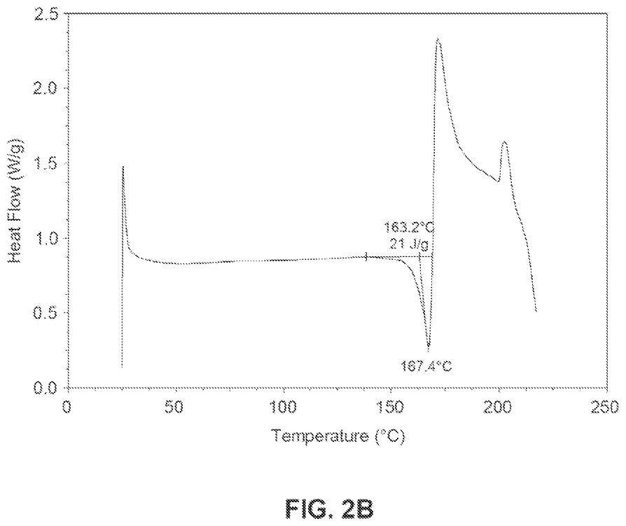

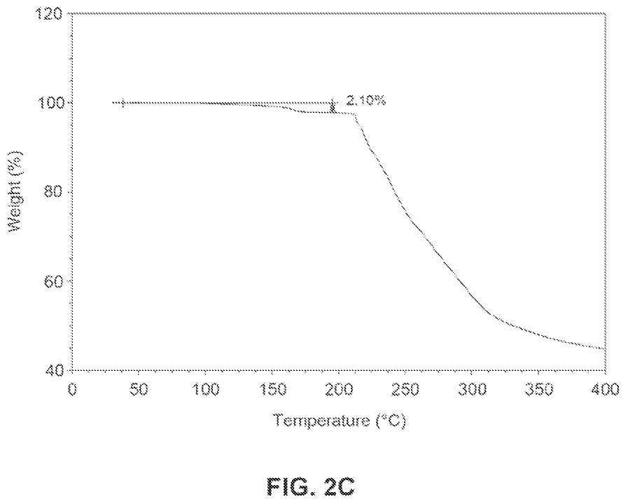

In another embodiment of the invention, Form 12 can be characterized by at least one of the following features (i)-(ii):
(i) at least one at least two, at least three, at least four, at least five, at least six, at least seven, at least eight, at least nine, at least ten, at least eleven or all of the X-ray powder diffraction peaks shown in Table 2 using CuKα radiation;
(ii) an X-ray powder diffraction pattern substantially similar to FIG. 2A using CuKα radiation.

TABLE 2

| Angle | d value |
|---|---|
| ° (2θ) ± 0.2 | Angstrom |
| 8.69 | 10.173 ± 0.012 |
| 10.27 | 8.606 ± 0.008 |
| 12.22 | 7.236 ± 0.006 |
| 13.21 | 6.699 ± 0.005 |
| 13.78 | 6.421 ± 0.005 |
| 14.10 | 6.277 ± 0.004 |
| 14.77 | 5.993 ± 0.004 |
| 15.01 | 5.898 ± 0.004 |
| 15.57 | 5.688 ± 0.004 |
| 16.08 | 5.506 ± 0.003 |
| 16.55 | 5.353 ± 0.003 |
| 16.75 | 5.288 ± 0.003 |
| 17.32 | 5.115 ± 0.003 |
| 17.51 | 5.060 ± 0.003 |
| 18.10 | 4.897 ± 0.003 |
| 18.64 | 4.757 ± 0.003 |
| 19.02 | 4.663 ± 0.002 |
| 19.55 | 4.537 ± 0.002 |
| 20.68 | 4.291 ± 0.002 |
| 21.17 | 4.193 ± 0.002 |
| 21.48 | 4.134 ± 0.002 |
| 22.11 | 4.017 ± 0.002 |
| 23.13 | 3.842 ± 0.002 |
| 23.65 | 3.759 ± 0.002 |
| 25.48 | 3.493 ± 0.001 |
| 25.90 | 3.438 ± 0.001 |
| 26.48 | 3.363 ± 0.001 |
| 27.21 | 3.274 ± 0.001 |
| 28.97 | 3.079 ± 0.001 |
| 29.51 | 3.024 ± 0.001 |

In an embodiment of the invention, Form 12 can be characterized by the differential scanning calorimetry profile (DSC) shown in FIG. 2B. The DSC graph plots the heat flow as a function of temperature from a sample, characterized by a having an endothermic peak of about 167.4° C., with an onset of about 163.2° C.

In an embodiment, Form 12 can be characterized by TGA profile, as shown in FIG. 2C, TGA analysis showed a 2.10% w/w mass loss between 110-210° C.

One embodiment is directed to crystalline Form 12, wherein the crystalline form is characterized by at least two of the following features (i)-(iv):
(i) at least one of the X-ray powder diffraction peaks shown in Table 2;
(ii) an X-ray powder diffraction pattern substantially similar to FIG. 2A;
(iii) a differential scanning calorimetry (DSC) profile having an endotherm range of about 162° C. to about 170° C., with an onset of about 163.2° C.; or
(iv) a thermogravimetric analysis (TGA) profile that shows a 2.10% w/w mass loss between 110-210° C.

### Pharmaceutical Compositions and Methods

Each of Crystal Form 9 and Crystal Form 12 is an inhibitor of NEDD8-activating enzyme (NAE), alone or in combination with other polymorphic forms of Compound 1.

One embodiment relates to a pharmaceutical composition comprising a Form 9 or Form 12 of Compound 1, and a pharmaceutically acceptable carrier or diluent. In an embodiment, a pharmaceutical composition is formed by admixing Form 9 of Compound 1 with a pharmaceutical carrier. In another embodiment, a pharmaceutical composition is formed by admixing Form 12 of Compound 1 with a pharmaceutical carrier. In another embodiment a pharmaceutical composition is formed by admixing Form 1 and Form 9 of Compound 1 with a pharmaceutical carrier.

The pharmaceutical compositions preferably are in a form suitable for administration to a recipient subject, preferably a mammal, more preferably a human. The term “pharmaceutically acceptable carrier” is used herein to refer to a material that is compatible with the recipient subject, and is suitable for delivering an active agent to the target site without terminating the activity of the agent. The toxicity or adverse effects, if any, associated with the carrier preferably are commensurate with a reasonable risk/benefit ratio for the intended use of the active agent.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, solubilizing agents, bioavailability modifiers and combinations of these.

Pharmaceutically acceptable carriers that may be used in these compositions include, but are not limited to, ion exchangers, alumina, aluminum stearate, lecithin, serum proteins, such as human serum albumin, buffer substances such as phosphates or carbonates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to one embodiment, the compositions are formulated for pharmaceutical administration to a mammal, preferably a human being. Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. Preferably, the compositions are administered orally, intravenously, or subcutaneously. The formulations of the invention may be designed to be short-acting, fast-releasing, or long-acting. Still further, compounds can be administered in a local rather than systemic means, such as administration (e.g., by injection) at a tumor site.

Pharmaceutical formulations may be prepared as liquid suspensions or solutions using a liquid, such as, but not limited to, an oil, water, an alcohol, and combinations of these. Solubilizing agents such as cyclodextrins including beta-cyclodextrin sulfobutylether and hydroxypropyl beta-cyclodextrin may be included. Other excepients present in the formulation include citric acid or sodium citrate. Pharmaceutically suitable surfactants, suspending agents, or emulsifying agents, may be added for oral or parenteral administration. Suspensions may include oils, such as but not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The pharmaceutical compositions may be orally administered in any orally acceptable dosage form including, but not limited to, capsules, tablets, aqueous suspensions or solutions. When aqueous suspensions are required for oral use, the active ingredient is combined with emulsifying and suspending agents. If desired, certain sweetening, flavoring or coloring agents may also be added. For oral administration in a capsule form, useful diluents include lactose and dried cornstarch. In the case of tablets for oral use, carriers that are commonly used include lactose and corn starch. Lubricating agents, such as magnesium stearate, are also typically added. Coatings may be used for a variety of purposes; e.g., to mask taste, to affect the site of dissolution or absorption, or to prolong drug action. Coatings may be applied to a tablet or to granulated particles for use in a capsule.

Alternatively, the pharmaceutical compositions may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The pharmaceutical compositions are particularly useful in therapeutic applications relating to disorders as described herein (e.g., proliferation disorders, e.g., cancers, inflammatory, neurodegenerative disorders). Preferably, the composition is formulated for administration to a patient having or at risk of developing or experiencing a recurrence of the relevant disorder being treated. The term “patient”, as used herein, means an animal, preferably a mammal, more preferably a human. Preferred pharmaceutical compositions of the invention are those formulated for oral, intravenous, or subcutaneous administration. However, any of the above dosage forms containing a therapeutically effective amount of a compound of the invention are well within the bounds of routine experimentation. In certain embodiments, the pharmaceutical composition may further comprise another therapeutic agent. Preferably, such other therapeutic agent is one normally administered to patients with the disorder, disease or condition being treated.

By “therapeutically effective amount” is meant an amount of compound or composition sufficient, upon single or multiple dose administration, to cause a detectable decrease in NAE enzyme activity and/or the severity of the disorder or disease state being treated. “Therapeutically effective amount” is also intended to include an amount sufficient to treat a cell, prolong or prevent advancement of the disorder or disease state being treated (e.g., prevent additional tumor growth of a cancer, prevent additional inflammatory response), ameliorate, alleviate, relieve, or improve a subject's symptoms of the a disorder beyond that expected in the absence of such treatment. The amount of Compound 1 required will depend on the particular composition given, the type of disorder being treated, the route of administration, and the length of time required to treat the disorder. It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated. In certain aspects where Compound 1 is administered in combination with another agent, the amount of additional therapeutic agent present in a composition of this invention typically will be no more than the amount that would normally be administered in a composition comprising that therapeutic agent as the only active agent. Preferably, the amount of additional therapeutic agent will range from about 50% to about 100% of the amount normally present in a composition comprising that agent as the only therapeutically active agent.

One embodiment of the disclosure relates to a method of inhibiting or decreasing NAE enzyme activity in a sample comprising contacting the sample with a solid state form of the disclosure, or a pharmaceutical composition of Form 9 or Form 12 of Compound 1. Another embodiment of the disclosure relates to a method of the disclosure. The sample, as used herein, includes, without limitation, sample comprising purified or partially purified NAE enzyme, cultured cells or extracts of cell cultures; biopsied cells or fluid obtained from a mammal, or extracts thereof, and body fluid (e.g., blood, serum, saliva, urine, feces, semen, tears) or extracts thereof. Inhibition of NAE enzyme activity in a sample may be carried out in vitro or in vivo, in cellulo, or in situ.

In another embodiment, the disclosure provides a method for treating a patient having a disorder, a symptom of a disorder, at risk of developing, or experiencing a recurrence of a disorder, comprising administering to the patient a solid state form of the disclosure, or a pharmaceutical composition of the disclosure. Treating can be to cure, heal, alleviate, relieve, alter, remedy, ameliorate, palliate, improve or affect the disorder, the symptoms of the disorder or the predisposition toward the disorder. While not wishing to be bound by theory, treating is believed to cause the inhibition of growth, ablation, or killing of a cell or tissue in vitro or in vivo, or otherwise reduce capacity of a cell or tissue (e.g., an aberrant cell, a diseased tissue) to mediate a disorder, e.g., a disorder as described herein (e.g., a proliferative disorder, e.g., a cancer, inflammatory disorder). As used herein, “inhibiting the growth” or “inhibition of growth” of a cell or tissue (e.g., a proliferative cell, tumor tissue) refers to slowing, interrupting, arresting or stopping its growth and metastases and does not necessarily indicate a total elimination of growth.

Disease applications include those disorders in which inhibition of NAE enzyme activity is detrimental to survival and/or expansion of diseased cells or tissue (e.g., cells are sensitive to NAE inhibition; inhibition of NAE activity disrupts disease mechanisms; reduction of NAE activity stabilizes protein which are inhibitors of disease mechanisms; reduction of NAE activity results in inhibition of proteins which are activators of disease mechanisms). Disease applications are also intended to include any disorder, disease or condition which requires effective cullin and/or ubiquitination activity, which activity can be regulated by diminishing NAE enzyme activity.

For example, methods of the disclosure are useful in treatment of disorders involving cellular proliferation, including, but not limited to, disorders which require an effective cullin-dependent ubiquitination and proteolysis pathway (e.g., the ubiquitin proteasome pathway) for maintenance and/or progression of the disease state. The methods of the disclosure are useful in treatment of disorders mediated via proteins (e.g., NFκB activation, p27^{Kip} activation, p21^{WAF/CIP1} activation, p53 activation) which are regulated by E1 activity (e.g., NAE activity, UAE activity, SAE activity). Relevant disorders include proliferative disorders, most notably cancers and inflammatory disorders (e.g., rheumatoid arthritis, inflammatory bowel disease, asthma, chronic obstructive pulmonary disease (COPD), osteoarthritis, dermatosis (e.g., atopic dermatitis, psoriasis), vascular proliferative disorders (e.g., atherosclerosis, restenosis) autoimmune diseases (e.g., multiple sclerosis, tissue and organ rejection)); as well as inflammation associated with infection (e.g., immune responses), neurodegenerative disorders (e.g., Alzheimer's disease, Parkinson's disease, motor neurone disease, neuropathic pain, triplet repeat disorders, astrocytoma, and neurodegeneration as result of alcoholic liver disease), ischemic injury (e.g., stroke), and cachexia (e.g., accelerated muscle protein breakdown that accompanies various physiological and pathological states, (e.g., nerve injury, fasting, fever, acidosis, HIV infection, cancer affliction, and certain endocrinopathies)).

The methods of the present disclosure are directed to treating diseases, disorders and conditions in which inhibition of NAE enzyme activity is detrimental to survival and/or expansion of diseased cells or tissue (e.g., cells are sensitive to NAE inhibition; inhibition of NAE activity disrupts disease mechanisms; reduction of NAE activity stabilizes protein which are inhibitors of disease mechanisms; reduction of NAE activity results in inhibition of proteins which are activators of disease mechanisms). The diseases, disorders and conditions are also intended to include those which require effective cullin and/or ubiquitination activity, which activity can be regulated by diminishing NAE enzyme activity.

Solid state forms of the disclosure, and pharmaceutical compositions of the disclosure are particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

In some embodiments, the cancer is a solid tumor. Non-limiting examples of solid tumors that can be treated by the methods of the invention include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma.

In some other embodiments, the cancer is a hematologic malignancy. Non-limiting examples of hematologic malignancy include acute myeloid leukemia (AML); chronic myelomonocytic leukemia (CMML), chronic myelogenous leukemia (CMIL), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

In some embodiments, a solid state form of the disclosure, or a pharmaceutical composition of the disclosure is used to treat a patient having or at risk of developing or experiencing a recurrence in a cancer selected from the group consisting of colorectal cancer, ovarian cancer, lung cancer, breast cancer, gastric cancer, prostate cancer, and pancreatic cancer. In certain preferred embodiments, the cancer is selected from the group consisting of lung cancer, colorectal cancer, ovarian cancer and hematologic cancers.

In some embodiments, the methods of the present disclosure further comprise administering an anti-cancer agent. As used herein, the term “anticancer agent” refers to any agent that is administered to a subject with cancer for purposes of treating the cancer. The administration of the further anti-cancer agent includes administration concurrently or sequentially with the combinations of the present disclosure. Alternatively, the further anti-cancer agent can be combined into one composition with the combinations of the present disclosure which is administered to the patient. In some embodiments, a solid state form of the disclosure, or a pharmaceutical composition of the disclosure are administered in conjunction with a therapeutic agent selected from the group consisting of cytotoxic agents, radiotherapy, and immunotherapy appropriate for treatment of proliferative disorders and cancer. Non-limiting examples of cytotoxic agents suitable for use in combination with a solid state form of the disclosure include: antimetabolites, including, e.g., capecitibine, gemcitabine, 5-fluorouracil or 5-fluorouracil/leucovorin, fludarabine, cytarabine, mercaptopurine, thioguanine, pentostatin, and methotrexate; topoisomerase inhibitors, including, e.g., etoposide, teniposide, camptothecin, topotecan, irinotecan, doxorubicin, and daunorubicin; vinca alkaloids, including, e.g., vincristine and vinblastin; taxanes, including, e.g., paclitaxel and docetaxel; platinum agents, including, e.g., cisplatin, carboplatin, and oxaliplatin; antibiotics, including, e.g., actinomycin D, bleomycin, mitomycin C, adriamycin, daunorubicin, idarubicin, doxorubicin and pegylated liposomal doxorubicin; alkylating agents such as melphalan, chlorambucil, busulfan, thiotepa, ifosfamide, carmustine, lomustine, semustine, streptozocin, decarbazine, and cyclophosphamide; thalidomide and related analogs including, e.g., CC-5013 and CC-4047; protein tyrosine kinase inhibitors, including, e.g., imatinib mesylate and gefitinib; proteasome inhibitors, including, e.g., bortezomib; antibodies, including, e.g., trastuzumab, rituximab, cetuximab, and bevacizumab; mitoxantrone; dexamethasone; prednisone; and temozolomide.

Other examples of agents which may be combined with a solid state form of the disclosure, or a pharmaceutical composition of the disclosureinclude anti-inflammatory agents such as corticosteroids, TNF blockers, Il-1 RA, azathioprine, cyclophosphamide, and sulfasalazine; immunomodulatory and immunosuppressive agents such as cyclosporine, tacrolimus, rapamycin, mycophenolate mofetil, interferons, corticosteroids, cyclophosphamide, azathioprine, methotrexate, and sulfasalazine; antibacterial and antiviral agents; and agents for Alzheimer's treatment such as donepezil, galantamine, memantine and rivastigmine.

### Synthetic Methods

In some embodiments, solid state forms of the disclosure are manufactured from the compound of formula (I) Form 1, which is synthesized from the compound of formula (II), by treating an ethanolic solution of the compound of formula (II) with an HCl solution in either ethanol or diethyl ether.

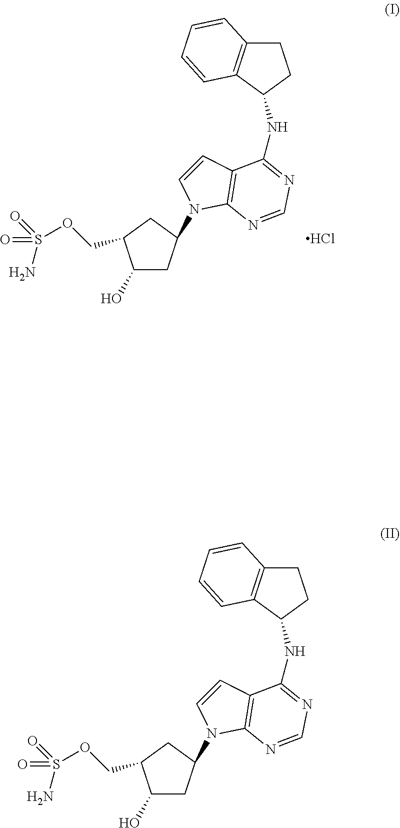 [SMILES: NS(=O)(=O)OC[C@@H]1C[C@@H](n2ccc3c(N[C@H]4CCc5ccccc54)ncnc32)C[C@@H]1O ; Cl ; NS(=O)(=O)OC[C@@H]1C[C@@H](n2ccc3c(N[C@H]4CCc5ccccc54)ncnc32)C[C@@H]1O]

The molarity of the HCl solution is about 0.9 M to about 1.3 M. When using the ethanolic HCl solution, the ethanolic solution of the compound of formula (II) is heated to a temperature of about 45° C. to about 55° C. before the HCl solution is added. When using the diethyl ether HCl solution, the ethanolic solution of the compound of formula (II) is stirred at a temperature of less than about 25° C. while the diethyl ether HCl solution is added.

Forms 2, 3A, 3B, 3C, 5 and 7 of Compound 1 are made by treating amorphous compound of formula (I) with the appropriate solvent. The crystalline form is generated by maturation using heat/cool cycles of the amorphous compound of formula (I) with the appropriate solvent. The crystalline form is generated by stirring the resulting slurry generated from the amorphous compound of formula (I) and the appropriate solvent, followed by evaporation of the excess solvent, or filtration of the crystalline material.

Form 1 converts to Form 9 via a metastable-anhydrate, Form 11, when slurried in methyl isobutyl ketone (MIBK) at room temperature.

Form 1 had been found to transform to Form 9 during heat cool cycle slurries in toluene. Form 1 was slurried in toluene at 50° C. to determine if the transformation was temperature dependent. After 2 days it was Form 9. In order to determine if the transformation went via Form 11, the experiment was repeated and monitored. After 23 hours, a mixture of Form 1 and Form 9 was observed, but not Form 11.

Form 1 was slurried in toluene at room temperature; however Form 1 did not convert to Form 9, but to a new polymorph, Form 12. This conversion was repeated multiple times, however the rate of conversion was dependent on the scale.

### EXAMPLES

### Abbreviations

DMF dimethylformamide
DSC differential scanning calorimetry
DMSO dimethylsulfoxide
EtOAc ethyl acetate
EtOH ethanol
MeOH methanol
MEK methyl ethyl ketone
THF tetrahydrofuran
HRMS high resolution mass spectrum
hr hours
min minutes
m/z mass to charge
MS mass spectrum
NMR nuclear magnetic resonance
RP LC-MS reverse phase liquid chromatography-mass spectrometry
RT room temperature
XRPD X-ray powder diffraction

### General Methods

Proton nuclear magnetic resonance spectra are obtained on a Varian Mercury 300 spectrometer at 300 MHz.

X-Ray Powder Diffractometry (XRPD): X-ray powder diffraction patterns for the samples are acquired on either:

Bruker D8 diffractometer using Cu Kα radiation (40 kV, 40 mA), θ-2θ goniometer, and divergence of V4 and receiving slits, a Ge monochromator and a Lynxeye detector. The instrument is performance checked using a certified Corundum standard (NIST 1976). The software used for data collection is Diffrac Plus XRD Commander v2.5.0, and the data are analysed and presented using Diffrac Plus EVA v 11.0.0.2 or v 13.0.0.2. Samples are run under ambient conditions. Approximately 30 mg of the sample is gently packed into a cavity cut into polished, zero-background (510) silicon wafer. The sample is covered by a Kapton film to prevent any contamination of the instrument during analysis, the film could also reduce evaporation of solvent contained in the material. The sample is rotated in its own plane during analysis. The data are collected at an angular range of 2 to 42° 2θ; with a step size of 0.05° 2θ; and a collection time of 0.5 s.step⁻¹.

Siemens D5000 diffractometer using Cu Kα radiation (40 kV, 40 mA), θ-θ goniometer, divergence of V20 and receiving slits, a graphite secondary monochromator and a scintillation counter. The instrument is performance checked using a certified Corundum standard (NIST 1976). The software used for data collection is Diffrac Plus XRD Commander v2.3.1 and the data are analysed and presented using Diffrac Plus EVA v 11.0.0.2 or v 13.0.0.2. Samples are run under ambient conditions as flat plate specimens. Approximately 35 mg of the sample is gently packed into a cavity cut into polished, zero-background (510) silicon wafer. The sample is rotated in its own plane during analysis. The data are collected at an angular range of 2 to 42° 2θ; with a step size of 0.05° 2θ; and a collection time of 4 s.step⁻¹.

The XRPD may also be collected on a Bruker D8Advance. The data are collected over an angular range of 2.9° to 29.6° 2θ in continuous scan mode using a step size of 0.05° 2θ and a step time of 2 seconds. The sample is run under ambient conditions and prepared as a flat plate specimen using powder without grinding. The control software is Diffrac Plus XRD Comander v 2.3.1, and the analysis software is Diffrac Plus EVA v 9.0.0.2. The samples are run either static or rotated under ambient conditions.

All values for XRPD data are ±0.2 2-Theta.

Differential Scanning Calorimetry (DSC): Differential scanning calorimetry (DSC) data are collected either on a Mettler DSC 823e equipped with a 50 position auto-sampler, or on a TA Instruments Q100 differential scanning calorimeter equipped with a 50 position auto-sampler, or on a TA Instruments Q200 differential scanning calorimeter. The energy and temperature calibration standard is indium. Samples are typically heated at a rate of 10° C. per minute between 25° C. and 250° or 300° C. A nitrogen purge flowing at 50 mL per minute is maintained over the sample during a scan. Between 0.5 mg and 3 mg of sample is analyzed. Samples are either crimped in a hermetically sealed aluminum pan with a pinhole to alleviate the pressure accumulated from the solvent vapor, or in a hermetically sealed aluminum pan without a pinhole.

### Example 1: DSC Analysis of Form 9

Example 1A: Analysis by Differential Scanning Calorimetry of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9

A small amount of Form 9 was sealed in a crimped DSC pan and heated from 25 to 300° C. at a constant ramp rate of 5° C./min under a nitrogen purge rate of 30 mL/min. Form 9 exhibited an endothermic event with an onset of 154.4° C. and maxima at 164.5° C. This was followed immediately by a second exothermic event at 179° C. Comparison to data obtained via thermogravimetric analysis indicated these events occurred concurrently with degradation of the sample.

Example 1B: Analysis by Differential Scanning Calorimetry of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9

Approximately 0.5 to 2 mg of Form 9 was placed in a pin-holed aluminum pan and heated from 25 to 200° C. at a constant ramp of 10° C./min under a nitrogen purge rate of 50 mL/min. Form 9 showed an exothermic even with an onset at 183.3° C. Comparison to data obtained via thermogravimetric analysis indicated these events occurred concurrently with degradation of the sample.

### Example 2: DSC Analysis of Form 12

Example 2A: Analysis by Differential Scanning Calorimetry of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12

Approximately 0.5 to 2 mg of Form 12 was placed in a pin-holed aluminum pan and heated from 25 to 200° C. at a constant rate of 10° C./min under a nitrogen purge rate of 50 mL/min. Form 12 exhibited an endothermic event, with an onset at 163.2° C. and melt at 167.4° C. This was immediately followed by multiple exothermic events and degradation of the material.

Example 2B: Analysis by Differential Scanning Calorimetry of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12

Approximately 1 to 2 mg of Form 12 was placed in a hermetically sealed aluminum pan and heated from 25 to 300° C. at a constant ramp rate of 10° C./min under a nitrogen purge rate of 50 mL/min. Form 12 was shown to have a small endothermic event was observed with an onset at 80.0° C. and maxima at 98.1° C. Form 12 exhibited a second endothermic event with an onset at 162.3° C. and melt at 168.5° C., immediately followed by a series of exothermic events that were indicative of degradation.

It is not uncommon to observe slight differences in analysis via differential scanning calorimetry for the same polymorph form. These differences can be attributed to a number of factors including, but not limited to, differences in pan preparation, thermal events occurring concurrently with or after the onset of degradation, the presence of residual solvents in differing amounts across batches, differences in heating ramp rates, and differences in nitrogen purge flows.

Thermal Gravimetric Analysis (TGA): Thermal gravimetric analysis (TGA) data are collected on either:
i) a Mettler TGA/SDTA 851e equipped with a 34 position autosampler, calibrated using certified indium. Typically 5-30 mg of each sample is loaded onto a pre-weighed aluminium crucible, and is heated at 10° C./min from ambient temperature to 250° C. A nitrogen purge flowing at 50 ml per minute is maintained over the sample;
ii) a TA Instruments Q500 thermal gravimetric analyzer, calibrated with Nickel/Alumel and running at a scan rate of 10° C. per minute. A nitrogen purge flowing at 60 mL per minute is maintained over the sample during measurements. Typically, 10 mg to 15 mg of sample is loaded onto a pre-tared platinum crucible.

### Example 3: TGA Analysis of Form 9

### Example 3A: Thermogravimetric Analysis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9

A small amount of Form 9 was placed in an open aluminum pan and heated from 25 to 300° C. at a constant ramp rate of 5° C./min under an atmosphere of nitrogen flow of 200 mL/min. Form 9 was found to have degradation occurring across multiple steps, with the initial degradation occurring at 151° C. and resulting in a w/w mass loss of 4.9%. A secondary degradation event was immediately observed following the first event, with initiation at approximately 179° C.

### Example 3B: Thermogravimetric Analysis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9

Approximately 5 to 10 mg of Form 9 was loaded onto a pre-tared aluminum DSC pan and heated at a constant ramp rate of 10° C./min from ambient temperature to 400° C. with a nitrogen flow of 60 mL/min maintained over the sample. Form 9 was shown to degrade over a range from approximately 150 to 243° C. and resulting in a w/w mass loss of 16.4%.

### Example 4: TGA Analysis of Form 12

### Example 4A: Thermogravimetric Analysis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12

Approximately 5 to 10 mg of Form 12 was loaded onto a pre-tared aluminum DSC pan and heated at a constant ramp rate of 10° C./min from ambient temperature to 400° C. with a nitrogen flow of 50 mL/min maintained over the sample. Form 12 was shown to lose mass over a range from approximately 40 to 196° C. and resulting in a w/w mass loss of 2.1%.

### Example 4B: Thermogravimetric Analysis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12

Approximately 5 to 10 mg of Form 12 was loaded onto a pre-tared aluminum crucible and heated at a constant ramp rate of 10° C./min from ambient temperature to 350° C. with a nitrogen flow of 60 mL/min maintained over the sample. Form 12 was shown to lose mass over a range from approximately 150 to 180° C. and resulting in a w/w mass loss of 1.2%.

It is not uncommon to observe slight differences in thermogravimetric analysis for the same polymorph form. These differences can be attributed to a number of factors including, but not limited to, the presence of residual solvents in differing amounts across batches, differences in heating ramp rates, differences in nitrogen purge rates, and differences in analysis data processing used to determine onset of mass loss.

### Example 5: Solid State Stability of Forms 1, 9, and 12

Solid-state stability studies of Forms 1, 9, and 12 were performed to assess the relative stability of new anhydrous Forms 9 and 12. Solid samples of each pattern were stored in open and closed glass vials at accelerated conditions (40° C./75% RH open and closed, and 60° C. closed). Samples were pulled after 1 and 3.5 weeks and analyzed for purity via HPLC (MTHD-0006487) and polymorphic form via XRPD for evidence of chemical degradation and solid form changes.

Table 1 and Table 2 below summarize the results of the study, which indicated that both Form 9 and Form 12 are stable when compared to Form 1 with respect to change in TAK-924 chemical purity (% Area) and physical form.

TABLE 1
HPLC Purity Results (% Area TAK-924) for Forms
1, 9, and 12 After Accelerated Storage

| | | 40° C./75% RH | | 40° C./75% RH | | | |
|---|---|---|---|---|---|---|---|
| | | Open (change | | Closed (change | | 60° C. (change | |
| | | from T-0) | | from T-0) | | from T-0) | |
| | Initial | T-6 | T-3.5 | T-6 | T-3.5 | T-6 | T-3.5 |
| Material | (T-0) | Days | Weeks | Days | Weeks | Days | Weeks |
| Compound Form 1 | 99.0 | 98.9 | 98.8 | 98.6 | 98.1 | 94.4 | 86.6 |
| | | (−0.1%) | (−0.2%) | (−0.4%) | (−0.9%) | (−5.6%) | (−13.4%) |
| Compound Form 9 | 99.4 | 99.4 | 99.3 | 98.6* | 99.4 | 99.2 | 98.2 |
| | | (0.0%) | (−0.1%) | (−0.8%) | (−0.2%) | (−0.2%) | (−1.2%) |
| Compound Form 12 | 99.0 | 98.9 | 98.9 | 99.0 | 98.8 | 98.5 | 97.0 |
| | | (−0.1%) | (−0.1%) | (0.0%) | (−0.2%) | (−0.5%) | (−2.0%) |

*Out of Trend

TABLE 2
XRPD Results for Forms 1, 9, and 12 after 3.5 Weeks of Accelerated Storage

| | 40° C./75% RH Open | | 40° C./75% RH Closed | | 60° C. | |
|---|---|---|---|---|---|---|
| | T-6 | T-3.5 | T-6 | T-3.5 | T-6 | T-3.5 |
| Material | Days | Weeks | Days | Weeks | Days | Weeks |
| Compound | Form 1 + | Form 1 + | Form 1 | Form 1 + | Form 1 | Form 1 + |
| Form 1 | Form 2 | Form 2 | | Form 2 | | Form 2 |
| Compound | Form 9 + | Form 9 + | Form 9 | Form 9 + | Form 9 | Form 9 + |
| Form 9 | Trace Form 2 | trace Form 2 | | trace Form 2 | | trace Form 2 |
| Compound | Form 12 | Form 12 | Form 12 | Form 12 | Form 12 | Form 12 |
| Form 12 | | | | | | |

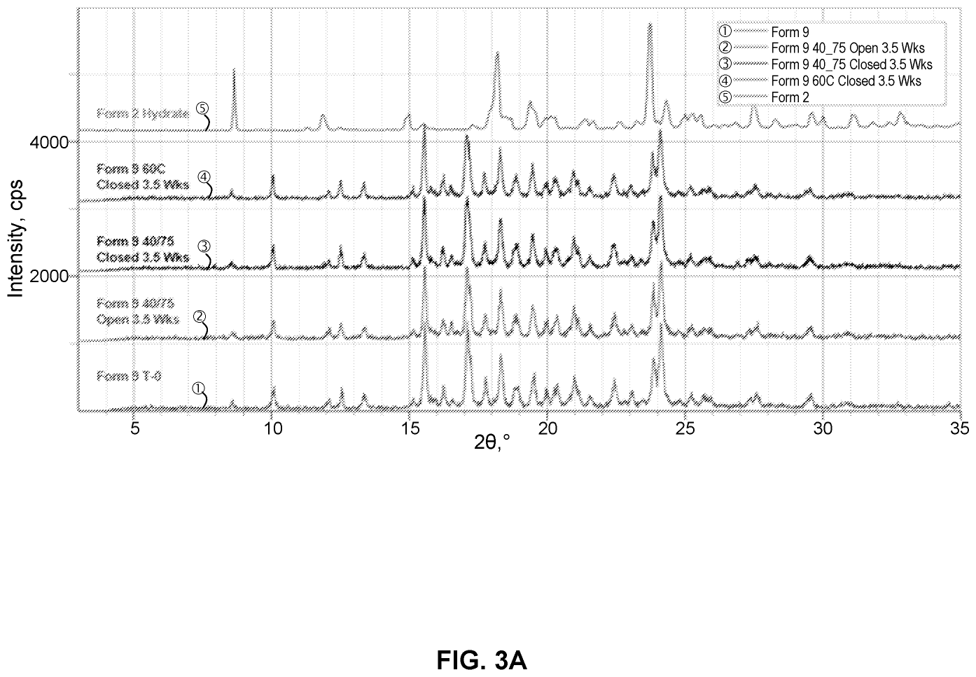

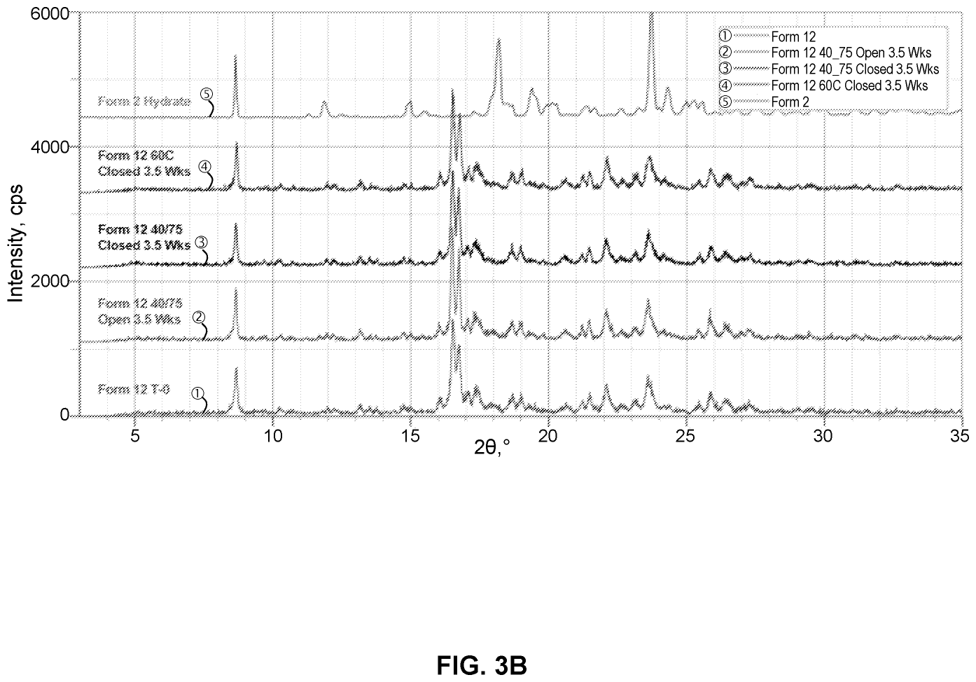

XRPD overlays of Form 9 and Form 12 after accelerated storage are shown in FIGS. 3A and 3B. Trace amounts of Form 2 are observed in Form 9. Form 12 shows no presence of Form 2 after accelerated storage. Overall, the stability data indicate that Forms 9 and Form 12 are chemically and physically stable.

### Example 6: Synthesis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 1

### Step 1: Preparation of (1S,2S,4R)-4-{4-[(1S)-2,3-dihydro-1H-inden-1-ylamino]-7H-pyrrolo[2,3-d]pyrimidin-7-yl}-2-(hydroxymethyl)cyclopentanol

A jacketed reactor was charged with (1S,2S,4R)-4-(4-chloro-7H-pyrrolo[2,3-d]pyrimidin-7-yl)-2-(hydroxymethyl)cyclopentanol (30.8 Kg, 115.05 mol), 2-butanol (198.5 Kg), (S)-(+)-1-aminoindane (16.95 Kg, 127.26 mol) and diisopropylethylamine (19.45 Kg, 150.50 mol). The mixture was heated to 55±5° C. and then moved to a mobile vessel. The reactor was then rinsed with 2-butanol (15.6 Kg) at 55±5° C. which was moved to the mobile vessel. The mobile vessel contents were then transferred to a pressure reactor and 2-butanol (51 L) was used to rinse the mobile vessel. The reaction mixture was then heated to 135±5° C. and adjusted to a pressure of 8 bar. The mixture was then stirred until reaction was complete by HPLC analysis. The mixture was cooled to 30±10° C. and transferred to a mobile vessel via a plate filter. The pressure reactor was rinsed with 2-butanol (43.1 L). The contents of the mobile vessel were then charged to a jacketed reactor via an in-line filter and the vessel rinsed with 2-butanol (39.2 Kg). The mixture was heated to 50±5° C. and concentrated under reduced pressure to about 50 L. The mixture was cooled to 20±5° C. and then dichloromethane (256 Kg) added over a period of about 3 hours. The mixture was stirred for a further 9.5 hours and then further cooled to 0±5° C. and stirred for about 4 hours. The solid product was isolated by filtration and washed with dichloromethane (82 Kg) at 0±5° C. The solids were then dried under reduced pressure at 40±5° C. to constant weight. A reactor was charged with water (371 Kg) and the dried solids and the mixture stirred at 20±5° C. for about 14.5 hours. The solid product was isolated by filtration and washed with water (371 Kg). The solids were then dried under reduced pressure at 50±5° C. to afford the title compound (32.4 Kg) as a white solid. ¹H NMR (300 MHz, DMSO, S): 8.15 (s, 1H), 7.71 (d, 1H), 7.07-7.29 (m, 5H), 6.61 (d, 1H), 5.88 (dd, 1H), 5.24-5.38 (m, 1H), 4.60 (d, 1H), 4.26-4.37 (m, 2H), 3.53-3.65 (m, 1H), 3.35-3.46 (m, 1H), 2.90-3.04 (m, 1H), 2.75-2.90 (m, 1H), 2.33-2.56 (m, 2H), 2.04-2.14 (m, 2H), 1.88-2.03 (m, 2H), 1.74-1.87 (m, 1H).

### Step 2: Preparation of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (II)

A jacketed reactor was charged with (1S,2S,4R)-4-{4-[(1S)-2,3-dihydro-1H-inden-1-ylamino]-7H-pyrrolo[2,3-d]pyrimidin-7-yl}-2-(hydroxymethyl)cyclopentanol (15.1 Kg, 41.43 mol), acetonitrile (86.2 Kg) and Sulfamating Reagent, prepared as described below (36.7 Kg, 83.4 mol). The mixture was heated to 46±6° C. and stirred until reaction was complete by HPLC analysis. The mixture was cooled to 20±5° C. and a solution of 0.5N aqueous hydrochloric acid (83.95 Kg) added maintaining a temperature below 25° C. The mixture is stirred vigorously until by-product consumption was complete by HPLC analysis. The layers were then separated and the aqueous phase extracted with tert-butyl methyl ether (56.2 Kg). The organic phases were combined and further tert-butyl methyl ether (18.1 Kg) was added. The organic phase was then washed with water (151.3 L). Acetonitrile (119.3 Kg) was added and the mixture then concentrated under reduced pressure to about 190 L. Further acetonitrile (77.6 Kg) was added and the mixture again concentrated under reduced pressure to about 190 L. The mixture was then cooled to −2.5±2.5° C. and concentrated hydrochloric acid (53.0 Kg) was added slowly maintaining a temperature below 5° C. The mixture was then warmed to 15±5° C. and stirred until reaction (deprotection) was complete by HPLC analysis. Water (151.1 L) was added maintaining a temperature below 25° C. followed by portion wise addition of sodium bicarbonate (46.0 Kg). The mixture was then heated at 20±5° C. for 1.5 hours. Ethyl acetate (137.1 Kg) was added and the mixture stirred for 1 hour. The layers were separated and the organic phase washed with water (150.7 L). The organic phase was then washed with 5% aqueous sodium chloride solution (2×159 Kg). The mixture was then concentrated under reduced pressure to about 100 L. A bed of acid washed activated charcoal (11.1 Kg) was equilibrated with ethyl acetate (48.3 Kg). The organic mixture was then passed through the charcoal bed (utilizing vacuum and pressure) and subsequent in-line filters (to remove any charcoal). The charcoal bed was then washed with ethyl acetate (245.2 Kg). The mixture was then concentrated to about 40 L under reduced pressure maintaining a temperature below 40° C. Ethyl acetate (87.7 Kg) was added and the mixture concentrated to about 40 L under reduced pressure maintaining a temperature below 40° C. Ethyl acetate (91.3 Kg) was added and the mixture concentrated to about 40 L under reduced pressure maintaining a temperature below 40° C. Ethyl acetate (88.6 Kg) was added and the mixture concentrated to about 40 L under reduced pressure maintaining a temperature below 40° C. Ethyl acetate (94.7 Kg) was added and the mixture concentrated to about 40 L under reduced pressure maintaining a temperature below 40° C. The mixture was then heated to 50±5° C. and dichloromethane (89.7 Kg) added at a rate to maintain a temperature of 50±5° C. The mixture was then seeded with the title compound (55 g) and further dichloromethane (502.6 Kg) added over 4 hours maintaining a temperature of 45±5° C. After stirring for a further 30 minutes the mixture was cooled to 20±5° C. and stirred for 16 hours. The mixture was then cooled to 2.5±2.5° C. and stirred for 8 hours. The solid product was isolated by filtration and washed with dichloromethane (1×50.1 Kg and 1×49.8 Kg) at 2.5±2.5° C. The solids were then dried under reduced pressure at ≤35° C. to afford the title compound (6.1 Kg) as a white solid. ¹H NMR (300 MHz, DMSO, S): 8.15 (s, 1H), 7.73 (d, 1H), 7.40 (s, 2H), 7.06-7.29 (m, 5H), 6.61 (d, 1H), 5.88 (dd, 1H), 5.26-5.42 (m, 1H), 4.90 (d, 1H), 4.26-4.35 (m, 1H), 4.14-4.25 (m, 1H), 3.95-4.07 (m, 1H), 2.90-3.04 (m, 1H), 2.75-2.89 (m, 1H), 2.62-2.74 (m, 1H), 2.40-2.55 (m, 1H), 1.97-2.18 (m, 3H), 1.83-1.96 (m, 2H).

### Preparation of Sulfamating Reagent

Chlorosulfonyl isocyanate (45.2 Kg, 319.4 mol) was added to toluene (194.2 Kg) and the resulting solution cooled to between about 0-6° C. A solution of tert-butyl alcohol (23.6 Kg, 318.4 mol) in toluene (48.0 Kg) was then added over a period of 90 minutes, maintaining a temperature of between about 0-6° C. The mixture was then stirred until consumption of tert-butyl alcohol was complete (approximately 80 minutes). A solution of triethylenediamine (DABCO, 71.4 Kg, 636.5 mol) in toluene (293.0 Kg) was then added to the mixture over a period of 2.5 hours, maintaining a temperature of between about 0-6° C. The mixture was then warmed to 20-25° C. and stirred for 8 hours. The solid product was isolated by centrifugal filtration under a nitrogen atmosphere and washed with toluene (180.8 Kg) and then tert butyl methyl ether (51.0 gallons) and spun until no further liquors were seen to be expelled (approximately 60 minutes). The solids were then further dried under vacuum to afford 132.9 Kg of the Sulfamating Reagent.

### Step 3: Synthesis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 1

A reactor was charged with ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (13.4 Kg, 30.2 mol) and 200-proof ethanol (106.2 Kg). The mixture was heated to reflux to afford a clear solution. The mixture was cooled to 50±5° C. and passed through a cartridge filter. 200 proof ethanol (8.9 kg) was used to rinse the filter. 1.27M hydrogen chloride in ethanol (10.2 Kg) was added via a cartridge filter at a rate to maintain a temperature of 50±5° C. The mixture was then seeded with Form 1 (67 g). Further 1.27M HCl (10.2 Kg) was added via a cartridge filter at a rate to maintain a temperature of 50±5° C. The mixture was then stirred at 50±5° C. for about 3 hours. The mixture was then cooled to 20±5° C. over about 3 hours and then stirred for about 2.5 hours. The solid product was then isolated by filtration and washed with 200-proof ethanol (1×20.4 Kg and 1×21.2 Kg). The solids were dried by aspiration on the filter until no supernatant was seen to be collected, and then further dried under reduced pressure at ≤30° C. to afford the title compound (12.2 Kg) as a white solid determined to be Form 1 by XRPD. 1H NMR (300 MHz, DMSO, S): 9.83 (s, 1H), 8.34 (s, 1H), 7.62 (s, 1H), 7.44 (s, 2H), 7.30 (m, 3H), 7.22 (t, 1H), 7.07 (s, 1H), 5.86 (dd, 1H), 5.42 (m, 1H), 4.32 (m, 1H), 4.21 (dd, 1H), 4.02 (dd, 1H), 3.04 (m, 1H), 2.88 (m, 1H), 2.67 (m, 2H), 2.15 (m, 2H), 2.08 (m, 2H), 1.94 (m, 1H).

### Alternative Preparation of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 1

To a reaction vessel is added ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate (1 equiv.) and ethanol (15 volumes with respect to input material) and the mixture is stirred at 20-25° C. 1.0M hydrogen chloride in ethanol (1 equiv. with respect to input material) is added at a rate as to maintain temperature at ≤25° C. The mixture is then stirred at 20±5° C. for a minimum of 4 hours. The solid product is isolated by filtration and washed with ethanol (2×2.5 volumes with respect to input material). The product is then dried by aspiration on the filter and then under reduced pressure at a temperature of 30±5° C. to give the title compound.

Example 7: Synthesis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 9 by interconversion from Form 1

Form 1 converts to Form 9, via a metastable-anhydrate, Form 11, when slurried in methyl isobutyl ketone at room temperature. Form 11 can be observed after stirring for 2-5 hours. If stirring is continued, it converts to Form 9. Form 9, when slurried in acetonitrile at room temperature, converts to Form 20 which is an acetonitrile solvate. Desolvation occurs extremely quickly such that Form 20 can only be observed in the XRPD if a dome has covered the sample. On desolvation, Form 1 is produced.

XRPD data for Form 9 is shown in FIG. 1A and Table 1; DSC and TGA data for Form 9 are both shown in FIG. 1B and FIG. 1C, respectively.

### Example 8: Synthesis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 12

A 20-ml vial was used with approximately 200 mg of Form 1 was slurried in 10 ml toluene and conversion to form 12 was effected in about 7-10 days.

XRPD data for Form 12 is shown in FIG. 2A and Table 2; DSC and TGA data for Form 12 are both shown in FIG. 2B and FIG. 2C, respectively.

### Example 9: Synthesis of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride Form 13

Form 1 was slurried in methyl isobutyl ketone (MIBK) at 5° C. and simultaneously Form 9 was also slurried in MIBK at 5° C. Form 1 converted to Form 11 after 3 days. However when a sample was removed and dried at 25° C., it was found to have converted to Form 9. The suspension was left for another 17 days, by which time it had converted to another new polymorph, which was identified as the MIBK solvate, named Form 13). The scale up of Form 13 was later carried out using seed crystals.

Having now fully described this invention, it will be understood by those of ordinary skill in the art that the same can be performed within a wide and equivalent range of conditions, formulations, and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

We claim:

1. A crystalline form of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride wherein the crystalline form is Form 9.

2. The crystalline form of claim 1, having an X-ray powder diffraction pattern comprising characteristic peaks plus or minus 0.2 degrees at 2θ angles of 15.60°, 17.16° and 18.30°

3. The crystalline form of claim 1, having an X-ray powder diffraction pattern comprising characteristic peaks plus or minus 0.2 degrees at 2θ angles of 15.60°, 17.16°, 18.30° and 24.10°.

4. The crystalline form of claim 2, wherein the X-ray powder diffraction pattern further comprises at least one of characteristic peaks plus or minus 0.2 degrees at 2θ angles of 12.55°, 20.39°, 22.45°, or 27.63°.

5. The crystalline form of claim 1, characterized by an XRPD pattern as shown in FIG. 1A.

6. The crystalline form of claim 1, wherein the crystalline form is characterized by the following features (i)-(ii):
i. at least three of the X-ray powder diffraction peaks shown in Table 1; and
ii. an X-ray powder diffraction pattern substantially similar to FIG. 1A

7. A crystalline form of ((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate hydrochloride wherein the crystalline form is Form 12.

8. The crystalline form of claim 7, having an X-ray powder diffraction pattern comprising characteristic peaks plus or minus 0.2 degrees at 2θ angles of 8.69°, 16.55°, 16.75 and 18.64°.

9. The crystalline form of claim 7, having an X-ray powder diffraction pattern comprising characteristic peaks plus or minus 0.2 degrees at 2θ angles of 8.69°, 16.55°, 16.75, 17.51°, 18.64°, 22.11° and 23.65°.

10. The crystalline form of claim 8, wherein the X-ray powder diffraction pattern further comprises at least one characteristic peaks plus or minus 0.2 degrees at 2θ angles of 19.02°, 21.17°, 25.9°, 26.48° or 28.97°.

11. The crystalline form of claim 7, characterized by an XRPD pattern as shown in FIG. 2A.

12. The crystalline form of claim 7, wherein the crystalline form is characterized by the following features):
(i) at least one of the X-ray powder diffraction peaks shown in Table 2; and
(ii) an X-ray powder diffraction pattern substantially similar to FIG. 2A

13. A pharmaceutical composition comprising any one of the compounds of claims 1-13, and a pharmaceutically acceptable carrier or diluent.

14. A method for treating cancer comprising the administration of a therapeutically effective amount of any one of the compounds according to claim 113 to a patient in need thereof.

15. The method of claim 15, the patient has or at risk of developing or experiencing a recurrence in a cancer selected from the group consisting of colorectal cancer, ovarian cancer, lung cancer, breast cancer, gastric cancer, prostate cancer, and pancreatic cancer.

16. The method of claim 15, wherein the cancer is multiple myeloma or lymphoma.
